(19) United States
(12) Patent Application Publication
(10) Pub. No.: US 2007/0073374 A1
(43) Pub. Date: Mar. 29, 2007

(54) Endoprostheses including nickel-titanium alloys

(76) Inventors: Steven F. Anderl, Forest Lake, MN (US); Kristopher H. Vietmeier, Monticello, MN (US)

Correspondence Address: FISH & RICHARDSON PC, P.O. BOX 1022, MINNEAPOLIS, MN, 55440-1022, US

(21) Appl. No.: 11/240,443

(22) Filed: Sep. 29, 2005

(51) Int. Cl. A61F 2/06

(52) U.S. Cl. 623/1.2

(57) ABSTRACT

Self-expanding endoprostheses, such as stents, have good fatigue resistance are disclosed.

### TECHNICAL FIELD

The invention relates to endoprostheses, such as stents.

### BACKGROUND

The body includes various passageways such as arteries, other blood vessels, and other body lumens. These passageways sometimes become occluded or blocked. For example, the passageways can be occluded by a tumor or restricted by plaque. When this occurs, the passageway can be reopened with a medical endoprosthesis. An endoprosthesis is typically a tubular member that is placed in a lumen in the body. Examples of endoprosthesis include stents, stent-grafts, and covered stents.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force. Alternately, self-expansion can occur through a material phase transition, induced by a change in temperature or by application of a stress.

To support a passageway open, endoprostheses are made of relatively strong materials formed into struts or wires. Examples of materials include stainless steel and Nitinol (a nickel-titanium alloy).

### SUMMARY

The invention relates to endoprostheses, such as stents, including a highly pure nickel-titanium alloy. The alloy has inclusions of small size, for example, in a low concentration. It is believed that small inclusions or the combination of small inclusions in a low concentration can provide the alloy with enhanced resistance to fatigue, such as alternating, cyclical fatigue. As a result, an endoprosthesis including the highly pure alloy can have enhanced fatigue resistance, for example, relative to an otherwise identical endoprosthesis including a less pure nickel-titanium alloy. Enhanced fatigue resistance can be particularly desirable when the endoprosthesis is implanted in a bodily vessel, such as the superficial femoral artery located behind the knee, that exposes the endoprosthesis to repeated stress (such as bending, flattening, stretching, and/or compressing).

In one aspect, the invention features an endoprosthesis including a generally tubular body adapted to self-expand from a first dimension to a second dimension to support a bodily vessel, the tubular body having an alloy including nickel and titanium, the alloy further including inclusions, wherein the largest inclusion is less than or equal to approximately 7 microns in length.

Embodiments of aspects of the invention may include one or more of the following features. The percent area concentration of inclusions is less than or equal to approximately 1%, for example, less than or equal to approximately 0.4%. The size of the largest inclusions is less than or equal to approximately 4 microns in length. The inclusions has an element selected from the group consisting of nitrogen, oxygen, and carbon. The tubular body has an oxidized layer less than or equal to approximately 100 angstroms, for example, less than or equal to approximately 30 angstroms. The alloy has from approximately 50.1 atomic percent to approximately 51.5 atomic percent of nickel. The endoprosthesis further includes a drug carried by the tubular body. The tubular body has an inner diameter of from approximately 5 mm to approximately 8 mm.

In another aspect, the invention features an endoprosthesis including a body adapted to self-expand from a first dimension to a second dimension and capable of maintaining the patency of a bodily vessel. The body includes an alloy having from approximately 50.1 atomic percent to approximately 51.5 atomic percent of nickel, and titanium, the alloy further having inclusions present in a percent area concentration of less than or equal to approximately 1%, the size of the largest inclusions being less than or equal to approximately 7 microns in length, wherein the tubular body has an oxidized layer less than or equal to approximately 100 angstroms. The inclusions can be present in an percent area concentration of less than or equal to approximately 0.4%.

In another aspect, the invention features a method including delivering an endoprosthesis comprising a generally tubular body into a bodily vessel, the tubular body having an alloy including nickel and titanium, the alloy further including inclusions, wherein the size of the largest inclusions is less than or equal to approximately 7 microns in length; and self-expanding the tubular body to support the bodily vessel.

Embodiments of aspects of the invention may include one or more of the following features. The bodily vessel is a superficial femoral artery or a carotid artery. The inclusions are present in an percent area concentration of less than or equal to approximately 1%, for example, less than or equal to approximately 0.4%. The size of the largest inclusions is less than or equal to approximately 4 microns in length. The inclusions has an element selected from the group consisting of nitrogen, oxygen, and carbon. The tubular body has an oxidized layer less than or equal to approximately 100 angstroms, for example, less than or equal to approximately 30 angstroms. The alloy has from approximately 50.1 atomic percent to approximately 51.5 atomic percent of nickel. The tubular body carries a drug. The tubular body has an inner diameter of from approximately 5 mm to approximately 8 mm.

As used herein, an “alloy” means a substance composed of two or more metals or of a metal and a nonmetal intimately united, for example, by being fused together and dissolving in each other when molten.

Other aspects, features and advantages will be apparent from the description of the preferred embodiments thereof and from the claims.

### DESCRIPTION OF DRAWINGS

FIG. 1 is a perspective view of an embodiment of a stent.

FIG. 2 is a flow chart of an embodiment of a method of making a stent.

### DETAILED DESCRIPTION

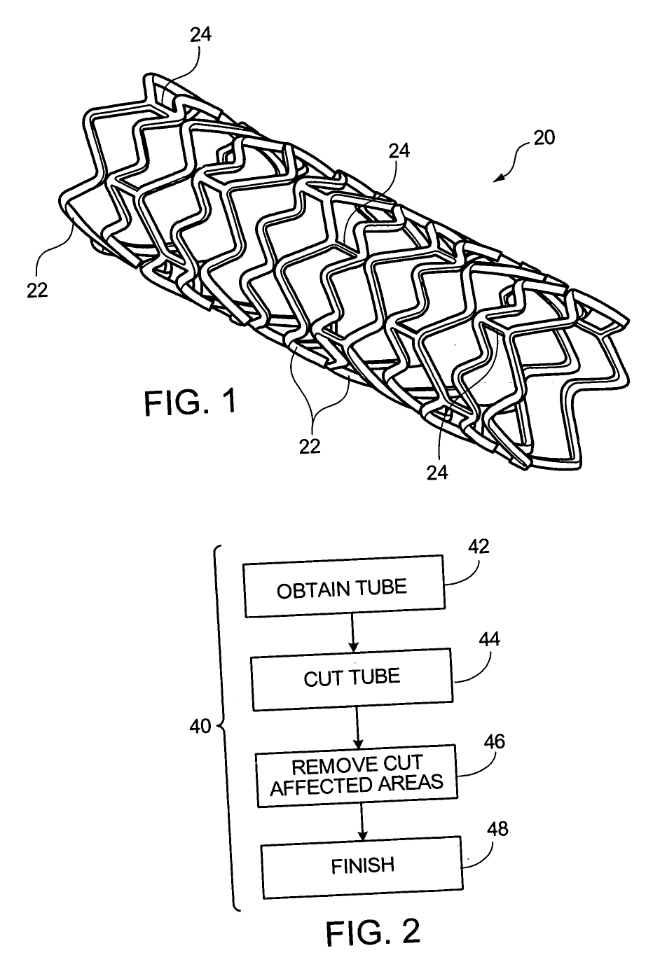

Referring to FIG. 1, a self-expandable stent 20 has the form of a tubular member defined by a plurality of bands 22 and a plurality of connectors 24 that extend between and connect adjacent bands. During use, bands 22 are expanded from an initial, small diameter to a larger diameter to contact stent 20 against a wall of a vessel, thereby maintaining the patency of the vessel. Connectors 24 provide stent 20 with flexibility and conformability that allow the stent to adapt to the contours of the vessel.

Stent 20 includes (e.g., is formed of) a highly pure, nickel-titanium alloy. In particular, the alloy has a low concentration of small-sized inclusions. As used herein, an inclusion is a region having a different chemical composition than the composition of the nickel-titanium alloy. For example, an inclusion may include nitrogen, carbon, and/or oxygen impurities in the form of titanium nitride or titanium oxide. The nickel-titanium alloy may include inclusions of different chemical compositions. Without wanting to be bound by theory, it is believed that the combination of small inclusions, present in a low concentration, provides the alloy with enhanced fatigue resistance. As a result, a stent including the alloy can better withstand fatigue when it is implanted in bodily vessel exposed to repeated stress. For example, when a stent is implanted in the superficial femoral artery located behind the knee, or in the carotid artery located in the neck, the stent can be exposed to bending forces, torsional forces, and/or compressive forces. By providing the stent with enhanced fatigue resistance, the risk of a band or a connector breaking, which can damage the bodily vessel or initiate a thrombosis, can be reduced.

As indicated above, the alloy has small-sized inclusions. The largest inclusions in a 500× scanning electron microscope (SEM) scan can be less than or equal to approximately 7 microns in length, for example, range from approximately 1 micron to approximately 7 microns in length. The inclusion size can be greater than or equal to approximately 1 micron, approximately 2 microns, approximately 3 microns, approximately 4 microns, approximately 5 microns, or approximately 6 microns; and/or less than or equal to approximately 7 microns, approximately 6 microns, approximately 5 microns, approximately 4 microns, approximately 3 microns, or approximately 2 microns. In some embodiments, the largest inclusion is less than or equal to approximately 1 micron. Cyclic fatigue performance can improve as the inclusion size is reduced and approaches 2 microns in drawn specimens. Even at the smaller size, fractures can initiate on inclusions, which may indicate that even smaller inclusions can further improve fatigue life. The size of the inclusions is determined by cross sectioning test specimens parallel to the drawing direction and measuring inclusion size using an SEM at 500 to 5000× magnification. The inclusions appear as black or grey discontinuities in the nickel-titanium alloy. The SEM is used to measure the sizes of the inclusions utilizing resident measurement features on the SEM. The largest inclusion is identified in a 500× scan area, and the largest inclusion is subsequently measured at 5000× magnification in which the largest major axis value of the inclusion is recorded. In measuring the largest inclusions, only whole inclusions are included, broken inclusions, voids and stringers are excluded.

The alloy can also have a low concentration of inclusions, expressed as a percent area concentration. The percent area concentration is the percentage of the total area occupied by the inclusions to the total area occupied by the inclusions and the nickel-titanium alloy (i.e., 100*[(total area of inclusions)/(total area of inclusions and nickel-titanium alloy)]). The percent area concentration of the inclusions can range from approximately 0.04% to approximately 1%, for example, less than or equal to approximately 0.25%. The percent area concentration of the inclusions can be greater than or equal to approximately 0.04%, approximately 0.1%, approximately 0.2%, approximately 0.3%, approximately 0.4%, approximately 0.5%, approximately 0.6%, approximately 0.7%, approximately 0.8%, or approximately 0.9%; and/or less than or equal to approximately 1%, approximately 0.9%, approximately 0.8%, approximately 0.7%, approximately 0.6%, approximately 0.5%, approximately 0.4%, approximately 0.3%, approximately 0.2%, or approximately 0.1%. The concentration of the inclusions can be determined by cross sectioning test specimens parallel to the drawing direction and measuring inclusion size using an SEM at 500× magnification. Inclusions appear as black or grey discontinuities in the nickel-titanium alloy. The percent area of inclusions can be determined by taking a digital SEM image at 500× magnification, using image analysis software to differentiate the inclusions from the nickel-titanium alloy, and then providing a pixel count of the inclusions compared to a pixel count of the nickel-titanium alloy. A pixel count ratio of the inclusions to the inclusions and nickel-titanium alloy is then used to calculate the percent area of the inclusions found in a 500× scan.

The chemical composition of the nickel-titanium alloy can also vary. In some embodiments, the alloy contains from approximately 50.1 atomic percent to approximately 51.5 atomic percent of nickel, with the remainder being titanium. For example, the alloy can contain from approximately 50.7 atomic percent to approximately 50.9 atomic percent of nickel, with the remainder being titanium. An example of a nickel-titanium alloy having the above low concentrations of small-sized inclusions is available from Nitinol Devices and Components (NDC) (Wayzata, Minn.) under the product name SE508 High Purity. NDC obtains its nickel-titanium alloy from Wah Chang (Albany, Oreg.), which uses vacuum arc remelting (VAR) to form the ingots of alloy having low carbon content.

To further enhance the fatigue resistance of stent 20, in some embodiments, the stent includes an oxidized outer surface layer of reduced thickness. The oxidized layer may include, for example, an oxidized form of the nickel-titanium alloy, such as a titanium oxide. A thick oxidized layer may appear blue, while a relatively thin oxidized layer may appear silver. In some embodiments, the oxidized layer has a thickness of les than approximately 100 angstroms, for example, less than approximately 30 angstroms. The thickness of the oxidized layer can be less than or equal to approximately 100 angstroms, approximately 90 angstroms, approximately 80 angstroms, approximately 70 angstroms, approximately 60 angstroms, approximately 50 angstroms, approximately 40 angstroms, approximately 30 angstroms, approximately 20 angstroms; and/or greater than or equal to approximately 5 angstroms, approximately 10 angstroms, approximately 20 angstroms, approximately 30 angstroms, approximately 40 angstroms, approximately 50 angstroms, approximately 60 angstroms, approximately 70 angstroms, approximately 80 angstroms, or approximately 90 angstroms. The thickness of the oxidized layer can be determined by Auger analysis.

Referring now to FIG. 2, a method 40 of making stent 20 is shown. Method 40 includes starting with a tube (step 42) including the alloy that makes up the tubular member of stent 20. As indicated above, a tube including a nickel-titanium alloy as described herein can be obtained from Nitinol Devices and Components (step 42). The tube is subsequently cut to form bands 22 and connectors 24 (step 44) to produce an unfinished stent. Areas of the unfinished stent affected by the cutting may be subsequently removed (step 46). The stent may be expanded and heat-set at temperatures known to those in the art, to form various finish diameters. The unfinished stent may be finished to form stent 20 (step 48).

Bands 22 and connectors 24 of stent 20 can be formed by cutting the tube (step 44). For example, selected portions of the tube can be removed to form bands 22 and connectors 24 by laser cutting, as described in U.S. Pat. No. 5,780,807, hereby incorporated by reference in its entirety. In certain embodiments, during laser cutting, a liquid carrier, such as a solvent or an oil, is flowed through the lumen of the tube. The carrier can prevent dross formed on one portion of the tube from re-depositing on another portion, and/or reduce formation of recast material on the tube. Other methods of removing portions of the tube can be used, such as mechanical machining (e.g., micro-machining), electrical discharge machining (EDM), and photoetching (e.g., acid photoetching).

In some embodiments, after bands 22 and connectors 24 are formed, areas of the tube affected by the cutting operation above can be removed (step 46). For example, laser machining of bands 22 and connectors 24 can leave a surface layer of melted and resolidified material and/or oxidized metal that can adversely affect the mechanical properties and performance of stent 20. The affected areas can be removed mechanically (such as by grit blasting or honing) and/or chemically (such as by etching or electropolishing). In some embodiments, the tubular member can be near net shape configuration after step 46 is performed. “Near-net size” means that the tube has a relatively thin envelope of material that is removed to provide a finished stent. In some embodiments, the tube is formed less than about 25% oversized, e.g., less than about 15%, 10%, or 5% oversized.

The unfinished stent is then finished to form stent 20 (step 48). The unfinished stent can be finished, for example, by electropolishing to a smooth finish. Since the unfinished stent can be formed to near-net size, relatively little of the unfinished stent need to be removed to finish the stent. As a result, further processing (which can damage the stent) and costly materials can be reduced. In some embodiments, about 0.0001 inch of the stent material can be removed by chemical milling and/or electropolishing to yield a stent.

Stent 20 can be of a desired shape and size (e.g., coronary stents, aortic stents, peripheral vascular stents, gastrointestinal stents, urology stents, and neurology stents). Depending on the application, stent 20 can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 4 mm to about 24 mm, for example, about 4 mm to about 14 mm. SFA stents can have an expanded diameter of from about 5 mm to about 8 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm.

In use, stent 20 can be used, e.g., delivered and expanded, using a catheter delivery system. The catheter delivery system is used to hold the stent in a radially compressed configuration during delivery of the stent to a target implantation site. At the implantation site, the catheter system is capable of allowing the stent to radially expand from the compressed configuration and releasing the stent, for example, by retracting an outer sheath. Catheter systems are described in, for example, Raeder-Devens, U.S. Pat. No. 6,726,712.

While a number of embodiments have been described above, the invention is not so limited.

As an example, while stent 20 is shown as being formed wholly of the alloy, in other embodiments, the alloy forms one or more selected portions of the medical device. For example, stent 20 can include multiple layers in which one or more layers include the alloy, and one or more layers do not include the alloy, e.g., a more radiopaque material such as platinum or gold. Stents including multiple layers are described, for example, in published patent application 2004-0044397, and Heath, U.S. Pat. No. 6,287,331.

Stent 20 can be a part of a covered stent or a stent-graft. For example, stent 20 can include and/or be attached to a biocompatible, non-porous or semi-porous polymer matrix made of polytetrafluoroethylene (PTFE), expanded PTFE, polyethylene, urethane, or polypropylene.

Stent 20 can include a releasable therapeutic agent, drug, or a pharmaceutically active compound, such as described in U.S. Pat. No. 5,674,242, U.S. Ser. No. 09/895,415, filed Jul. 2, 2001, and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

In some embodiments, a stent can be formed by fabricating a wire including the alloy, and knitting and/or weaving the wire into a tubular member. Examples of stents are described in Heath, U.S. Pat. No. 6,287,331, and Mayer, U.S. Pat. No. 5,800,511.

All references, patents, and applications referred to herein are incorporated by reference in their entirety.

Other embodiments are within the claims.

What is claimed is:

1. An endoprosthesis comprising a generally tubular body adapted to self-expand from a first dimension to a second dimension to support a bodily vessel, the tubular body comprising an alloy comprising nickel and titanium, the alloy further comprising inclusions, wherein the largest inclusion is less than or equal to approximately 7 microns in length.

2. The endoprosthesis of claim 1, wherein the percent area concentration of inclusions is less than or equal to approximately 1%.

3. The endoprosthesis of claim 1, wherein the size of the largest inclusions is less than or equal to approximately 4 microns in length.

4. The endoprosthesis of claim 1, wherein the inclusions are present in a percent area concentration of less than or equal to approximately 0.4%.

5. The endoprosthesis of claim 1, wherein the inclusions comprise an element selected from the group consisting of nitrogen, oxygen, and carbon.

6. The endoprosthesis of claim 1, wherein the tubular body comprises an oxidized layer less than or equal to approximately 100 angstroms.

7. The endoprosthesis of claim 1, wherein the tubular body comprises an oxidized layer less than or equal to approximately 30 angstroms.

8. The endoprosthesis of claim 1, wherein the alloy comprises from approximately 50.1 atomic percent to approximately 51.5 atomic percent of nickel.

9. The endoprosthesis of claim 1, further comprising a drug carried by the tubular body.

10. The endoprosthesis of claim 1, wherein the tubular body has an inner diameter of from approximately 5 mm to approximately 8 mm.

11. An endoprosthesis comprising a body adapted to self-expand from a first dimension to a second dimension and capable of maintaining the patency of a bodily vessel, the body comprising an alloy comprising from approximately 50.1 atomic percent to approximately 51.5 atomic percent of nickel, and titanium, the alloy further comprising inclusions present in a percent area concentration of less than or equal to approximately 1%, the size of the largest inclusions being less than or equal to approximately 7 microns in length, wherein the tubular body comprises an oxidized layer less than or equal to approximately 100 angstroms.

12. The endoprosthesis of claim 11, wherein the inclusions are present in an percent area concentration of less than or equal to approximately 0.4%.

13. A method, comprising:
delivering an endoprosthesis comprising a generally tubular body into a bodily vessel, the tubular body comprising an alloy comprising nickel and titanium, the alloy further comprising inclusions, wherein the size of the largest inclusions is less than or equal to approximately 7 microns in length; and
self-expanding the tubular body to support the bodily vessel.

14. The method of claim 13, wherein the bodily vessel is a superficial femoral artery.

15. The method of claim 13, wherein the bodily vessel is a carotid artery.

16. The method of claim 13, wherein the inclusions are present in an percent area concentration of less than or equal to approximately 1%;

17. The method of claim 13, wherein the size of the largest inclusions is less than or equal to approximately 4 microns in length.

18. The method of claim 13, wherein the inclusions are present in a percent area concentration of less than or equal to approximately 0.4%.

19. The method of claim 13, wherein the inclusions comprise an element selected from the group consisting of nitrogen, oxygen, and carbon.

20. The method of claim 13, wherein the tubular body comprises an oxidized layer less than or equal to approximately 100 angstroms.

21. The method of claim 13, wherein the tubular body comprises an oxidized layer less than or equal to approximately 30 angstroms.

22. The method of claim 13, wherein the alloy comprises from approximately 50.1 atomic percent to approximately 51.5 atomic percent of nickel.

23. The method of claim 13, wherein the tubular body carries a drug.

24. The method of claim 13, wherein the tubular body has an inner diameter of from approximately 5 mm to approximately 8 mm.
